(19) United States
(12) Patent Application Publication
(10) Pub. No.: US 2022/0351638 A1
(43) Pub. Date: Nov. 3, 2022

(54) STIMULATION OF BRAIN PATHWAYS AND RETINAL CELLS FOR VISION TRAINING

(71) Applicant: Eye Quix, LLC, San Jose, CA (US)

(72) Inventor: Gloria Wu, San Jose, CA (US)

(21) Appl. No.: 17/733,913

(22) Filed: Apr. 29, 2022

Related U.S. Application Data
(60) Provisional application No. 63/182,680, filed on Apr. 30, 2021.

(51) Int. Cl. G09B 19/00; G06T 13/00; G06T 11/20; G06T 11/00; A61B 3/032

(52) U.S. Cl. CPC G09B 19/003; G06T 13/00; G06T 11/203; G06T 11/001; A61B 3/032; G06T 2200/24

(57) ABSTRACT

A vision stimulation platform is designed for stimulation of brain pathways and retinal cells for vision training and enhancement. Modules of eye exercises are provided to a user, where each module includes an ordered sequence of eye exercises to be performed by the user. The eye exercises comprise one or more screens showing an animated display for the user to view for a time period less than a threshold time (e.g., 10 seconds). The animated display has a color, movement, or pattern designed to stimulate a specific visual pathway of the brain or the retina of the user, and the set of displays is designed to achieve a purpose (e.g., eye relaxation, vision precision, stroke treatment, etc.). At least one of the eye exercises comprises an interactive portion for the user to interact with one or more items on the screen to test the motor cortex of the user.

### CROSS-REFERENCE TO RELATED APPLICATION

This application claims the benefit of U.S. Provisional Patent Application 63/182,680, entitled “Stimulation of Brain Pathways and Retinal Cells for Vision Training and Enhancement” and filed Apr. 30, 2021, the contents of which are incorporated by reference.

### BACKGROUND

### 1. Field of Art

This disclosure relates to vision training and enhancement and particularly to a system and method designed for stimulation of brain pathways and retinal cells for vision training and enhancement.

### 2. Description of the Related Art

Vision restoration or enhancement of vision has been a long sought-after goal in ophthalmology and optometry. Specifically, the goal relates to how to restore functional vision to those patients with eye disease or normal people with minor changes in vision. Those with nearsightedness (myopia) wish get rid of their glasses or those with reading glasses (presbyopia) wish to get rid of their reading glasses. Those recovering from stroke affecting the visual pathway wish to regain their vision for driving or regain the activities of daily living. Similarly, those with closed head trauma, traumatic brain injury, post concussive syndrome patients wish for restoration of their attention span and their vision prior to their accident. Conventional treatments for all of these vision restorations are lacking.

Children with amblyopia (lazy eye) have long been treated with the wide accepted practice of patching the stronger eye so that the weaker eye can “regain” vision (occlusion therapy). The mechanism of action has been taught to all ophthalmologists that the brain is forced to use the visual input from the weaker (amblyopic or lazy eye). In adults, the treatment of amblyopia may mean recovery of vision with the same simple occlusion therapy (patching the stronger eye forcing the weaker eye to act). The conventional patching practice, however, is somewhat cumbersome and limited in its ability to treat the condition. Eyedrops that are being used in some countries for myopia in children. There is not widespread use, however, since these eyedrops are generally expensive ($100 to $150 per bottle per month), they are generally not covered by insurance, and they generally require a specialty compounding pharmacy with special sterilizing labs within its facility.

There are also some situations where it would be beneficial to have special acuity in vision for a select purpose and/or for a limited period of time. For example, for athletes, those in the military sharpshooting battalions, or those who operate sensitive instruments for warfare, it may be extremely beneficial to have superb vision for a few seconds at a time. As another example, those people who want to pass the vision part of their driver's license test, a test which takes only a few seconds, may want a vision boost. However, conventional technologies are not designed to provide these types of limited time vision boosts.

And those who use their computer screens 8 hours a day for work and then go back to the computer, smartphone or tablet for entertainment, movies, there is a need for relaxation of their eyes, retina, and focusing mechanisms to maintain their visual sharpness and attention spans for their work the following day. Similarly, relaxation techniques could enhance further productivity using the computer screen by enabling enhanced visual concentration and accuracy with software coding or computer reading. Again, however, conventional technologies are not designed to address this need.

### SUMMARY

A vision stimulation platform (system and method) is designed to stimulate various brain pathways along with the retina cells to engage vision enhancement and vision training for a wide range of applications. For example, the system can provide benefits to persons who want to see better, or to relax their tired eyes, relieve headaches from computer use or excessive time doing close work, eye patients with eye diseases, brain injury patients, and stroke patients. The platform includes a set of games or exercises designed specifically to enhance vision, train the eyes, relax the eyes, among other end goals, all within a single platform or application. The system is not limited to targeting a specific problem, but instead provides eye training as a whole. The eye training can include different sections or segments designed to target specific eye problems or needs. The platform, however, is able to provide simultaneous stimulation of the entire vision pathway and its relationship to the brain, including in a systematic manner that provides total brain stimulation, and then stimulation to different parts of the visual pathway, including for retina, color, attention focusing, and balance. The platform also provides a motor component for some eye exercises, including reaching, touching, saccades, and pursuit. Examples of benefits provided include for treatment of myopia (low to high grades of myopia), amblyopia, stroke rehabilitation, traumatic brain injury treatment, post concussive disorder, adult amblyopia of undetermined origin, presbyopia, etc. Other benefits include for athletes to improve vision with fast balls or reaction time, for persons with tired eyes due to computer vision syndrome or eye fatigue from too much focusing, for preparing for a long drive by refocusing the eye-brain so that attention span can be revived, quick improvement of vision just for particular purposes, including passing a motor vehicle eye test, precise target practice, military applications, sports competitions, etc.

The vision stimulation platform includes a set of games or exercises for a user to perform. These can be provided via a computer-implemented system, such as a mobile application or web application that the user can access on a computer or mobile device. Each game or exercise provides a user interface display to a user that is animated and/or interactive, requiring the user to perform an eye exercise by watching the screen and possibly performing one or more actions (e.g., tapping certain icons or features on a screen). Each exercise provides a display is provided for a finite period of time that is less than some threshold (e.g., 10 seconds or less) that is selected to optimize for the particular exercise being performed. In some cases, the exercise may cycle between different displays during the 10 seconds. The platform also provides modules or sets of exercises, including animated or interactive displays provided to a user in a particular order, the order specifically selected to stimulate different parts of the visual pathway in the brain and in the retina. Each exercise or display screen may stimulate a different visual pathway in the brain and/or in the retina. The platform may target the entire brain and different aspects of the brain as the software application continues. The retina pathways can be added into each test or exercise in a particular sequence.

The visual stimulation platform provides benefits in many areas. One example in which vision treatment has been efficacious is for stroke patients. Brain plasticity or neuroplasticity have been shown to occur after stroke. The occipital lobe which can be damaged in stroke and vision loss can recover with more synaptic pathway growth of the extra-striate cortex. See References 1, 2, 3, 4, 5, 6, and 7. Vision restoration can also spontaneously occur after brain and retinal damage. See Reference 8. The use of vision activation has been part of the treatment modalities for amblyopia since the 1930's. Researchers such as Bernhard Sabel in Switzerland (Referance 8) have found that residual vision can occur in patients with brain damage from stroke or vision lost with glaucoma. Neuroplasticity focuses on the surviving brain structures at the site of the problem and the total brain network. The visual system plasticity is described in the normal developing brain for young children. In the last decade, in the aging brain, perceptual learning can increase eye function for the elderly. See References 3, 4, 5, and 8. There is research indicating that amblyopia shows decreased activity in the occipital lobe and the striate cortex in adults (Referance 15). In children, the occipital lobe, bilateral frontal lobes, and temporal lobes are affected (Referance 16). Adaptation of the brain to various stimuli for the weaker eye in amblyopia can improve vision (Referance 17). Thus, the visual stimulation platform provides a way to increase eye function for patients, thereby helping stroke patients and other patients whose visual cortex (occipital lobe) are affected. This is just one example of how the visual stimulation platform can be used. Numerous others are described throughout. The platform uniquely provides multiple different types of eye exercises in a single application that can be used to treat various conditions or otherwise enhance vision.

Described herein is a method comprising: receiving, in a mobile application on a client device, a selection from a user of a module of eye exercises to stimulate a visual pathway of the user; providing, for display to the user on the mobile application, a first eye exercise in an ordered sequence of exercises, the first eye exercise comprising one or more screens showing an animated display for the user to view for a time period less than a threshold time, the animated display having one or more of a color, movement, or pattern that is designed to stimulate a first visual pathway of the brain or the retina of the user; receiving an indication that the user has completed the first eye exercise; following receipt of the indication of completion of the first eye exercise, providing for display to the user on the mobile application, a second eye exercise in an ordered sequence of exercises, the second eye exercise comprising one or more screens showing an animated display for the user to view for a time period less than a threshold time, the animated display having one or more of a color, movement, or pattern that is designed to stimulate a second visual pathway of the brain or the retina of the user; receiving an indication that the user has completed the second eye exercise; upon the user completing the module of eye exercises, confirming to the user the completion of the module; wherein at least one of the first and second eye exercises comprises an interactive portion for the user to interact with one or more items on the screen to test the motor cortex of the user, and wherein the ordered sequence of exercises is ordered specific to a particular end goal of the user in conducting the eye exercises.

Also described herein is a method comprising: in response to a selection from a user of a module of eye exercises to stimulate a visual pathway of the user, receiving, from a server at a mobile application on a client device, a first eye exercise in an ordered sequence of exercises, the first eye exercise comprising one or more screens showing an animated display for the user to view for a time period less than a threshold time, the animated display having one or more of a color, movement, or pattern that is designed to stimulate a first visual pathway of the brain or the retina of the user; receiving an indication that the user has completed the first eye exercise; following receipt of the indication of completion of the first eye exercise, receiving, from a server at a mobile application on a client device, a second eye exercise in an ordered sequence of exercises, the second eye exercise comprising one or more screens showing an animated display for the user to view for a time period less than a threshold time, the animated display having one or more of a color, movement, or pattern that is designed to stimulated a second visual pathway of the brain or the retina of the user; receiving an indication that the user has completed the second eye exercise; upon the user completing the module of eye exercises, confirming to the user the completion of the module; wherein at least one of the first and second eye exercises comprises an interactive portion for the user to interact with one or more items on the screen to test the motor cortex of the user, and wherein the ordered sequence of exercises is ordered specific to a particular end goal of the user in conducting the eye exercises.

Additionally, the methods described herein may be stored as instructions on a non-transitory, computer-readable medium, such that the instructions, when executed by a processor, cause the processor to perform the methods described herein. Such a computer-readable medium may be part of a computer-implemented system that further comprises a processor.

### BRIEF DESCRIPTION OF DRAWINGS

FIG. 1 is a diagram of a computing environment for providing visual stimulation to a user, according to embodiments.

FIGS. 2A, 2B, 2C, 2D, 2E, 2F, 2G, 2H, and 2I are example user interfaces, according to embodiments.

FIGS. 3A, 3B, 3C, 3D, 3E, 3F, 3G, 3H, 3I, 3J, 3K, 3L, 3M, 3N, and 30 are example user interfaces, according to embodiments.

FIG. 4 is a flow chart illustrating a sequence of eye exercises for normal people, according to embodiments.

FIG. 5 is a flow chart illustrating a sequence of eye exercises for people wishing to have precise vision, according to embodiments.

FIG. 6 is a flow chart illustrating a sequence of eye exercises for people with brain injuries, amblyopia (pediatric or adult), or brain conditions, according to embodiments.

FIG. 7 is a flow chart illustrating a sequence of eye exercises for elderly people, according to embodiments.

FIG. 8 is a flow chart illustrating a sequence of eye exercises for all people, according to embodiments.

FIG. 9 is a flow chart illustrating a sequence of eye exercises for people with eye fatigue, according to embodiments.

FIG. 10 is a chart illustrating certain results from an experiment performed with a visual stimulation system, in accordance with some embodiments.

FIG. 11 is a chart illustrating certain results from an experiment performed with a visual stimulation system, in accordance with some embodiments.

FIG. 12 is a chart illustrating certain results from an experiment performed with a visual stimulation system, in accordance with some embodiments.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### I. System Overview

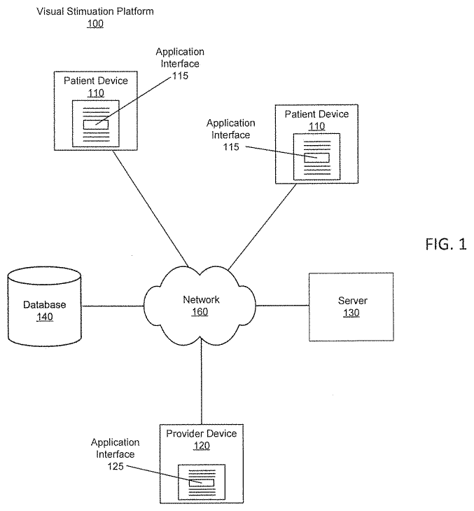

FIG. 1 is a diagram of a computing environment for a visual stimulation platform 100 according to one embodiment. The computing environment includes the server 130, one or more patient (client) devices 110, one or more providers 120, and a database 140, each connected to a network 160. Some embodiments of the computing environment may have additional, fewer, or different components than the ones described herein. For example, the patient devices 110 can represent thousands or millions of devices for patients (e.g., patient mobile devices) that interact with the platform in locations around the world. Similarly, the provider device 120 can represent thousands or millions of devices of health providers (e.g., mobile phones, laptop computers, in-provider-office recording devices, etc.). In some cases, a single provider may have more than one device that interacts with the platform 130. The functions can be distributed among the components in a different manner than described in FIG. 1.

A patient (also referred to as a user) can interact with the visual stimulation platform 100 through the patient device 110. A patient device 110 can be a personal or mobile computing device, such as a smartphone, a tablet, a notebook computer, a virtual assistant device (e.g., a GOOGLE HOME or AMAZON ECHO), a headset, head-mounted device, or other device for virtual reality, augmented reality, or mixed reality device. The patient device 110 is a computing device with data processing and data communication capabilities that is capable of receiving inputs from a patient and graphically presenting data to a patient (e.g., a graphics display). In some embodiments, the patient device 110 executes a client or mobile application that uses an application programming interface (API) to communicate with the visual stimulation system 100 through the network 160. The client or mobile application 115 of the patient device 110 can present information received from or generated by the visual stimulation system 100 or the server 130 on a user or application interface 115, such as reminders provided to the user to conduct certain eye exercises at particular times throughout the day or during a time period. In addition, the client or mobile application 115 may present data generated locally by the application 115 without any connection to the server 130. In some embodiments, there is no server 130 associated with the application 115.

Application 115 provides a user interface that is displayed on a screen of the patient device 110 and allows a patient to input commands to control the operation of the application 115. The application 115 enables patients to select different eye exercises perform, interact with the screen, provide an augmented reality display with an eye exercise overlaid on a view of a patient's room or other objects. The application 115 may be coded as a proprietary application configured to operate on the native operating system of the patient device 110. The application 125 may also be coded as a web page, series of web pages, or content otherwise coded to render within an internet browser. In addition to providing the user interface, application 115 may also perform some data processing using the resources of patient device 110 before sending the processed data through the network 160. Patient data sent through the network 160 is received by the server 130 where it is analyzed and processed for storage and retrieval in conjunction with a database 140.

Similarly, a provider device 120 is a computing device with data processing and data communication capabilities that is capable of receiving input from a provider. The provider device 120 is configured to present a patient's medical history or medically relevant data (i.e., a display screen), eye health history, medical health history, etc. Some of this data may be stored and retrieved from database 140 or from an internal database of the provider 120 or from a database accessed by a network of providers. The above description of the functionality of the patient device 110 also can apply to the provider device 120. The provider device 120 can be a personal device (e.g., phone, tablet) of the provider, a medical institution computer (e.g., a desktop computer of a hospital or medical facility), etc. In addition, the provider device 120 can include a device that sits within the provider office. The provider device 120 may also present information to medical providers or healthcare organizations via a software or mobile application 125 similar to the application described with reference to patient device 110, also having an application interface 125. In some embodiments, providers 120 are computer servers including one or more databases storing health information or physiological data of users. The platform 100 may use an API to communicate with providers 120, which may be associated with third party applications. In some embodiments, the provider 120 is not a part of the platform 100

In an embodiment, a provider 120 provides electronic medical record (EMR) data. The EMR data includes, for example, medical histories, doctor and hospital visits, medications, allergies, immunizations, medical test results, billing information, demographic data, etc., of the users. The EMR data may be updated over time based on information provided by health care providers such as a user's personal care physician or a nurse, or based on information provided by a user herself or himself.

Patient devices 110 and providers 120 can communicate with the servers 130 via the network 160, which may comprise any combination of local area and wide area networks employing wired or wireless communication links. In one embodiment, the network 160 uses standard communications technologies and Internet protocols. For example, the network 160 includes communication links using technologies such as the Internet, 3G, 4G, BLUETOOTH®, or WiFi. In some embodiments, all or some of the communication links of the network 160 may be encrypted.

### II. Example Visual Stimulation Interfaces

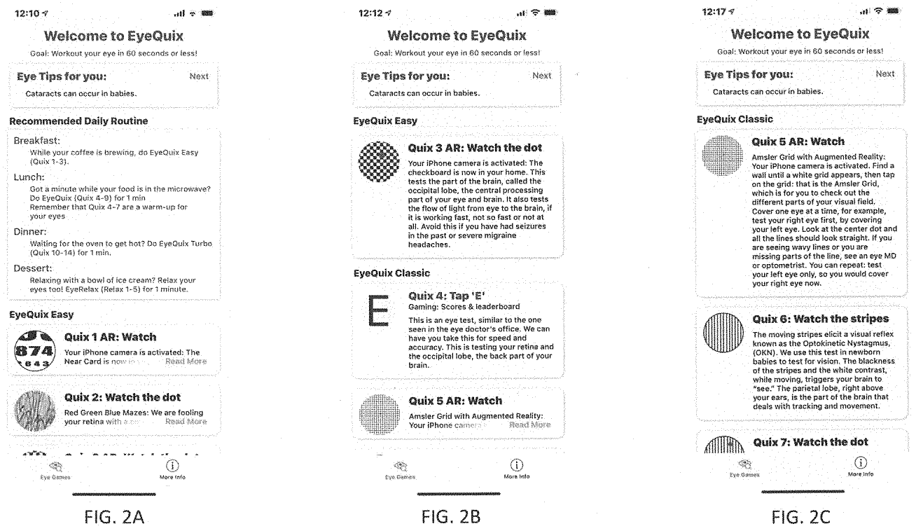

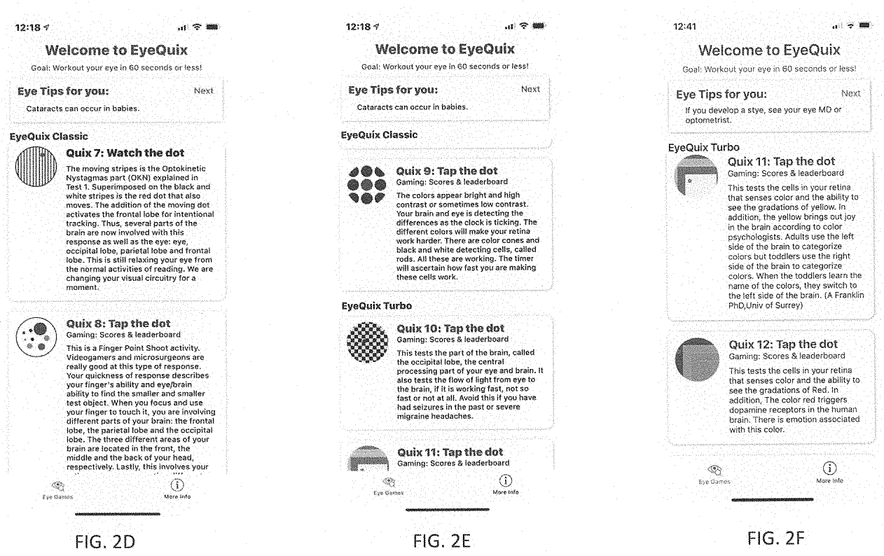

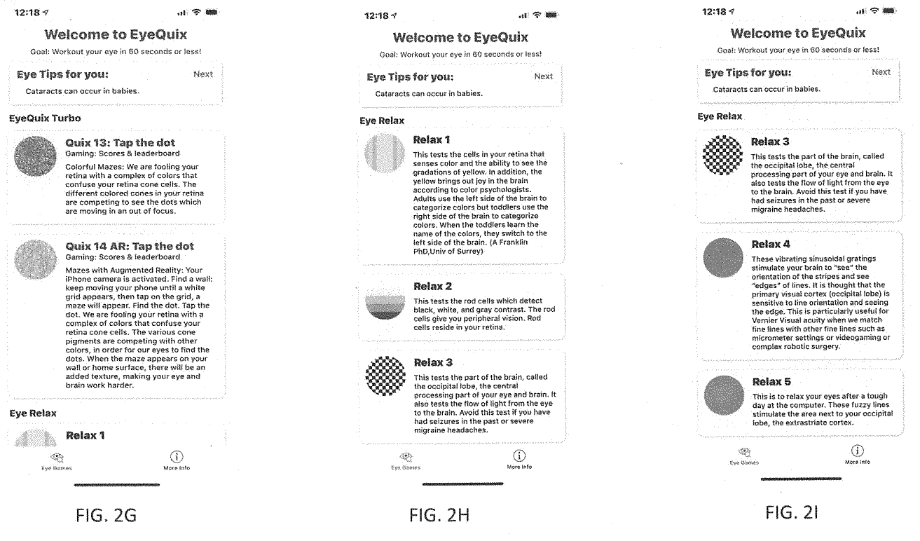

FIGS. 2A, 2B, 2C, 2D, 2E, 2F, 2G, 2H, and 2I provide a number of different user interface examples for the application 115 provided on the patient device 110, according to some embodiments. Specifically, these illustrate the home screen which the user can scroll through different exercise modules to find an exercise within a module to perform. The home screen also includes a number of recommendations and “eye tips” for the user recommending certain exercises or actions for vision enhancement or training.

In the examples, the application is named “EyeQuix”, and the views shown by the figures are home screens from which the user can select a module or an exercise or game to perform. It includes a number of different modules or sets of exercises that are provided on display screens to a use. In this example, the modules have names, such as Easy, Classic, Turbo, and Relax. Within each module, there is a set of one or more exercises, typically two to four exercises. In this example, each exercise or game is referred to as a “Quix” or in some cases a “Relax,” and these exercises are numbered. The Easy module includes Quix 1-3. The Classic module includes Quix 4-9. The Turbo module includes Quix 10-14. The Relax module includes Relax 1-5. These are just a few examples. There can be numerous modules, each having a different series of exercises. The exercises may all be unique or in some cases there are some overlapping exercises across different modules. Each module may have a different purpose or target, and each module has different characteristics, which are described in more details in the figures that follow.

II. a. Patterns, Movement, and Color

Each exercise involves providing a user with a display or graphic user interface 115 on the patient device 110. The display includes an animation that the user watches for a period of time. The animation may be a pattern presented on a screen that is stationary or is moving across a screen (e.g., horizontal or vertical stripes, checkerboard, dots, boxes, spots of different sizes, shapes, irregular lines, etc.).

The pattern may move across the screen in a constant or varying manner. The pattern may scroll across the screen in different directions (e.g., horizontally, vertically, diagonally, irregularly), it may flash or pulse on a screen, and it may move in one direction only or vary directions during the course of the exercise.

The particular pattern chosen for a given exercise is relevant because the viewing of the user of the pattern and the particular way that it moves in the display affects the visual stimulation and is designed to stimulate different parts of the visual pathway in the brain and/or in the retina.

In addition to which pattern is chosen for a given exercise and how it is animated on the screen, the colors presented on the screen affect the visual stimulation provided to the user, as well. A given pattern may be displayed in black and white, in multiple colors, with a specific selection of colors, with one color over black and white (e.g., a colored dot over a black and white pattern background), etc. In some cases, the colors chosen are designed to stimulate different parts of the visual pathway in the brain and/or in the retina.

Some embodiments of the exercises are interactive, where the user interacts with the display screen in some manner. For example, there may be an object, such as a dot, moving to different locations on the screen. The user may be asked to follow that dot with the user's eyes. The dot may become larger or smaller as it moves, may change in color, or may be displayed over a changing or moving patterned background (e.g., a red dot moving around on a screen over a black checkerboard pattern scrolling across a screen). The user may also be asked to tap the dot each time it moves to test motor ability of the user and/or reaction speed. The application may track certain parameters like the speed of the user in tapping the dot or how many times the user correctly taps the right location of the screen containing the dot. As another example, the user may be presented with areas of different colors that are similar, and the user may be asked to select the one area of color that is different from the rest. The user may do this across numerous screens showing different colors, and the application may track how many times the user correctly selects the different color. Each different interaction exercise is designed to test certain aspects of the user's eyes, and to stimulate different parts of the visual pathway in the brain and/or in the retina.

In some embodiments, the tests include music or other sounds that can affect the user's performance of the exercise or mental state. For example, in the Relax exercises, the application can provide relaxing music for the user to listen too, to further enhance the eye exercises. In other example, sounds or music is used as an additional “distraction” to challenge the user's attentional capabilities when doing the games.

II. b. Time period

The period of time during which the user performs the exercise is relevant to the stimulation of the user's visual pathways. The user may perform each test for a limited period of time, such as from 1-60 seconds, or from 1-5 minutes, or other range of time. For example, a given exercise may run for 1 second, 5 seconds, 10 seconds, 15 seconds, 20 seconds, 30 seconds, 60 seconds, or other values. The time period can be less than a threshold time, such as less than 10 seconds, or it can be 10 seconds or less. In addition, for a given exercise, the user may view multiple different screens, each one displayed for a short period of time. For example, a user may interact with each screen and when the interaction is complete, the screen may switch to a different version of the screen (e.g., switch colors or patterns). When a user selects an exercise to begin, the user may be presented with one or more displays for the exercise, and when the exercise is complete, the user may be notified that it has been completed. For example, the user may do a 10 second exercise and be notified at the end of the 10 second period. The user may then be presented with the option to perform the next exercise in the module. The user may continue in this manner until all exercises in a module are completed, after which the user may close the application or start a new module, or repeat the same module. A module might include, for example, five separate 10-second exercises.

II. c. Sequence

The sequence of exercises is relevant to the visual stimulation provided to the user. When a user performs a module, the exercises can be provided to the user in a specific sequence and may be numbered according to the sequence. The sequence may be designed to stimulate a first portion of a visual pathway initially with a first exercise, then to stimulate a second portion of a visual pathway with a second exercise, and so forth with a third, fourth, fifth, or any number of exercises. Some may affect the same visual pathway. Some may circle back to the same visual pathway after affecting others in between.

As one example, in a given module, a first exercise might test the occipital lobe and the cerebellum, along with the striate cortex. A second exercise might test the retinal cone cells to see colors. A third exercise might test the rod cells to discern different colors and contrast. A fourth exercise might test the motor cortex of the brain to touch the user interface. One or more of these exercises might be combined into a single exercise but be tested across a series of screens in the same exercise or by tested in single screen all at once. An additional exercise might return to testing the occipital lobe and striate pathway, along with various other areas. Finally, an eye chart may be presented in an exercise to test visual acuity. This eye chart can also be presented at the beginning and end of the module to see how vision is affected by the tests performed.

The particular sequence can be designed for a particular purpose or end goal, e.g., to treat stroke, traumatic brain injury, amblyopia, to improve fine vision, to relax tired eyes, etc. The order of the exercises and the particular exercises and lengths of time are optimized for the particular purpose.

II. d. Augmented Reality

In some exercises, augmented reality functionality is applied using the camera of a user's phone that shows the environment of the user, and presents certain patterns on the environment (e.g., checkerboard pattern, grid pattern, etc.). In some embodiments, other types of reality such as mixed or virtual reality are used. As an alternative to a mobile phone, the user may wear a head-mounted display such as a virtual reality headset, and may perform the exercises within the virtual or augmented reality environment. In one embodiment, the augmented reality games ask the user to move in space to engage the cerebellum such that the user is doing total body movements rather than just eye movements. In other embodiments, the augmented reality games move objects around in space in front of the user or toward the user, and the user takes certain actions on the objects. For example, an augmented reality game executed by a head-mounted display may allow the user to click on objects to cause the appearance of the object to change. Similarly, an augmented reality game may cause an object to react, deflect, or otherwise be controlled or affected in response to a user looking or gazing at the object. Where a headset or head-mounted display is worn, the headset allows for the visual field of one of a user's eyes to be covered or blocked, similar to wearing an eye patch to cover one eye during eye exercises performed in a medical office. The headset may allow for the visual field of a user's eye to be covered or blocked through a physical blocking of the display to the user, or by allowing an augmented reality game to cause the display to one of the user's eyes to be shut off or reduced. The headset also may blur the visual field of one eye, including controlling the level of blurring such that one eye can be presented with higher or lower levels of blurring of the visual field. The headset also allows for different images or video to be presented to each eye, including providing different colors, shapes, types of objects, speeds of movement of the objects, directions of movement of the objects, etc.

In pediatric and adult amblyopia, there has been research in using videogames and computer software programs for dichoptic training, i.e., bilateral eye stimulation with moving objects and shapes such as the Gabor patches. However, the training has been at least one hour per session. Dichoptic training means that the weaker eye or the “lazy eye” is presented with images while simultaneously blurring the stronger eye, all at the same time. The use of video games and iPad games have been tried binocularly and with eye patching. Dichoptic training is accepted among eye care physicians who treat amblyopia. However, it is challenging to have young children wear an eye patch, especially for a longer period of time, as the children tend to remove the patch. With a head-mounted display, the headset can cover the eye without requiring the child to wear a patch, and the child engages with a game in the virtual or augmented reality environment to provide bilateral eye stimulation with moving objects/shapes.

The presentation of the visual stimuli to the “lazy eye” in amblyopic patients has also been tried in small clinical studies with some positive benefit. The system described here allows the “lazy eye” to be stimulated while easily patching the good eye using, for example, the head-mounted display. Lastly, the presentation of the visual stimuli to both eyes may be useful as well since the stimulation is directed at the visual cortex and not the eye itself. For example, the exercises referred to as Quix 6, 7, 8, or 9 can provide visual stimuli to both eyes when presented, for example, on the screen of a mobile phone.

The augmented reality function works the cerebellum, the striate cortex, the frontoparietal cortex and the motor cortex as well as the insula, which facilitates “attentional” looking. Augmented reality requires the amblyopic patient to use the hands and body to find the image, and thus stimulate those interrelated visual pathways that were blunted in development. This feature of augmented reality works total brain, eyes and body movement, thus it provides valuable visual stimulation and training.

II. e. Example Exercises

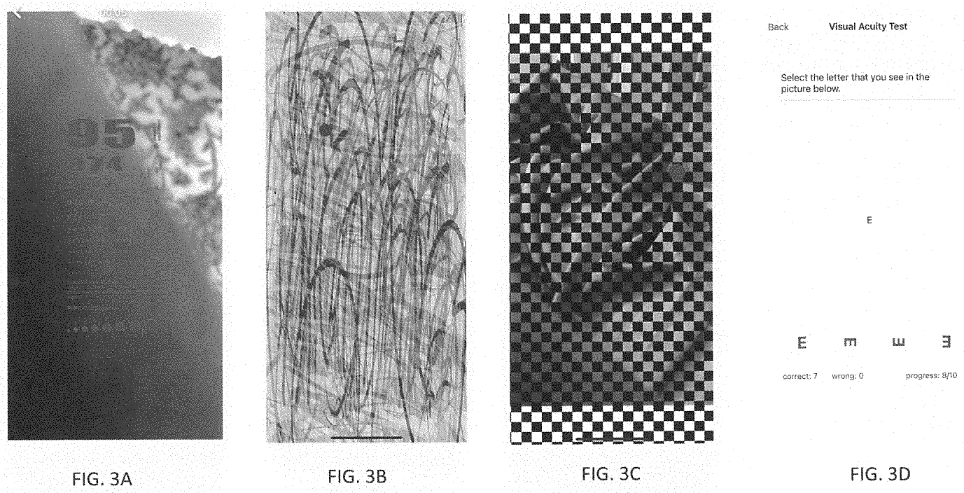

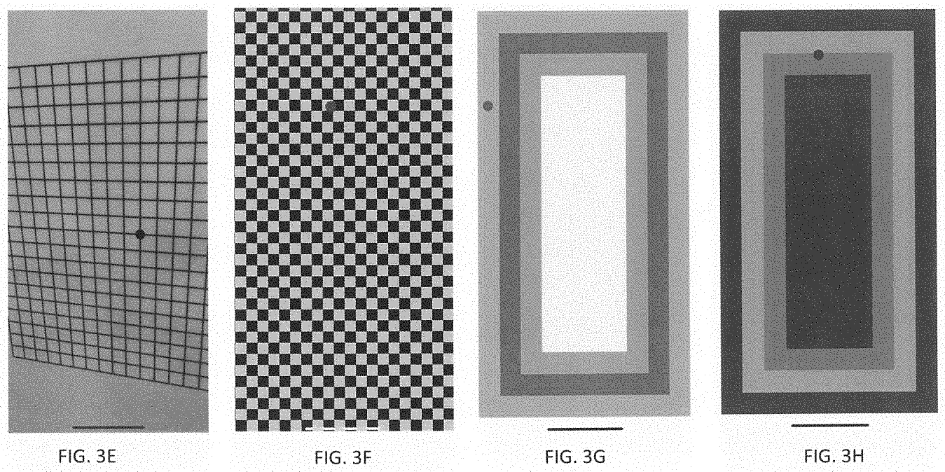

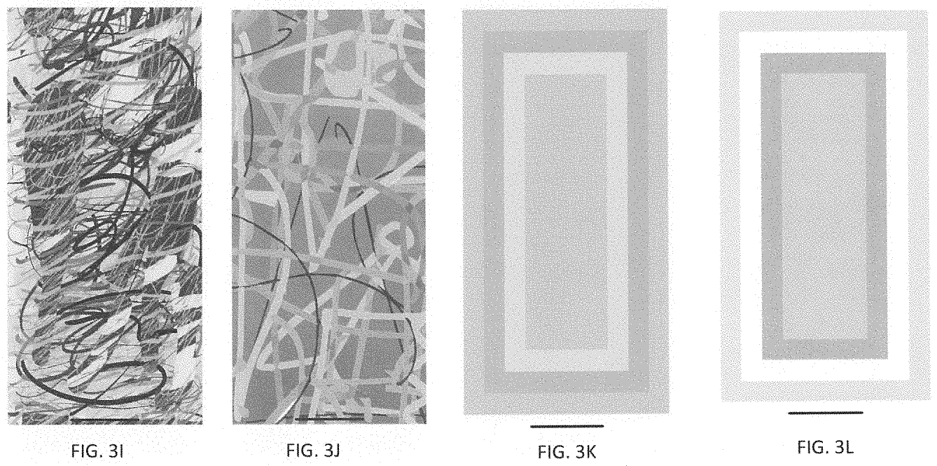

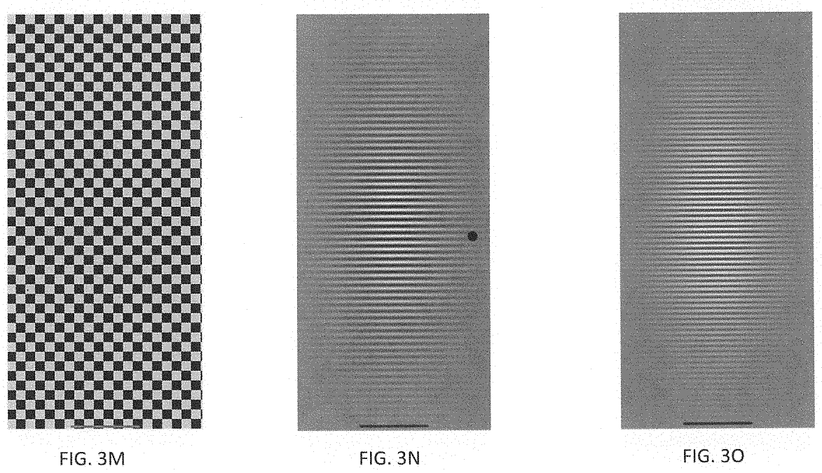

FIGS. 3A, 3B, 3C, 3D, 3E, 3F, 3G, 3H, 3I, 3J, 3K, 3L, 3M, 3N, and 3O provide a number of different user interface examples for the application 115 provided on the patient device 110. These figures show the example screens provided to the user during different eye exercises. These are just a few examples. These examples use the timed intervals of 10 seconds or approximately 10 seconds for each game to stimulate yet not saturate the user's attention span, though other timed intervals can be used.

FIGS. 3A-3C show exercises of the Easy module in this example. FIG. 3A is an eye test chart or “near card” that is suspended in space (Quix 1). An augmented reality (AR) view of the user's own environment (e.g., a view of the user's home environment through the mobile device camera) is provided as a background, and the near card is overlaid on the background. The near card is also animated, and moves in space (e.g., spins around) in front of the background. The user can also move around the mobile device to view the environment moving around in the background behind the overlaid near card. The near card can be displayed in different colors each time the user performs the exercise. This tests the occipital lobe and the cerebellum and the striate cortex next to the occipital lobe, challenging the eyes and cerebellum to read the numbers.

FIG. 3B (Quix 2) is a watch the dot (or other animated object/shape) exercise where the dot appears in different locations on the screen in a different color and/or different size for each appearance (it could also change in shape or other features). The user's eyes watch the dot as it moves around the screen wherever it appears. The dot is presented on a colorful, busy background of curved or swirled lines. The curved lines are composed of numerous colors (e.g., 2, 3, 4, 5 or more colors, such as blue, green, purple, yellow, pink orange), displayed over a solid background (e.g., yellow or blue background). The lines can be different widths, and be irregularly positioned to form a swirled, pattern. This is a color confusion exercise that tests the retina cone cells to see the colors, and then uses the rod cells to discern color contrast, and also uses the motor cortex of the brain to touch the dot. It uses the insula part of the brain (parietal lobe which is the side part of the brain). The red color cones are competing against the green color cones as the dots are moving in and out of the maze.

FIG. 3C (Quix 3) is an augmented reality (AR) exercise where a checkerboard pattern (e.g., in black) appears over the user's own environment (e.g., inside the user's home as viewed through the user's device camera) and scrolls across the screen. The user watches the dot (or other animated object/shape) as it moves around on this background, where the dot appears in different locations on the screen in a different color and/or different size for each appearance. The user can also move around the mobile device to view the environment moving around in the background behind the overlaid pattern. This checkerboard is the visual evoked response (VER) testing the occipital lobe, the striate pathway, located in the superior colliculus, cerebellum, reticular formation and the extraocular cortex, next to the occipital lobe. The dot that moves tests the fixation part of visual muscles, cranial nerves III, IV, VI. The camera on the mobile device is open, and thus there is an AR view of the environment, and this challenges the rods and cones in the retina to ignore those colors as the background. The ability to ignore and choose one primary object (the moving dot) engages executive control in the frontal lobe. This also tests the flow of light from eye to the brain, and whether it is working fast, not so fast, or not at all.

FIG. 3D (Quix 4) is a visual acuity chart, and is an example of an exercise in the Classic module. The visual acuity chart shows the “E” in a first position in a first screen, then rotated in a second screen to a new position, and also smaller in the second screen. This continued with the “E” getting smaller and smaller and rotating position each time. The user selects which is the correct position, and the app tracks the user's score, which is displayed on the screen. This tests occipital lobe (back part of brain) and the integrity of the retina to function, and the macula, to see the smallest E.

FIG. 3E (Quix 5) is another example of an exercise in the Classic module, where an Amsler Grid is displayed on an AR view of the user's environment. The user can use a mobile device camera positioned to view a surface (e.g., a wall) in the user's own environment and a grid (e.g., a white grid of boxes) appears on the surface. The user taps on the grid and it is replaced with an Amsler Grid (e.g., black lined grid)which allows the user to check out different parts of the user's visual field. The user can cover one eye at a time, for example, to test each eye, and can look at the center dot, and all of the lines should appear straight. If the lines appear wavy or portions appear to be missing, there may be an issue and the user should seek a professional opinion. This exercise uses the cerebellum to move the Amsler Grid and the occipital lobe to track it

Tests 10-14 (Turbo module) are designed for people with better vision, such as gamers, athletes, normal people, or young teens. FIG. 3F (Quix 10) is a tap the dot (or other animated object/shape) exercise on a black checkerboard with a colored background that scrolls across the screen. The background may be gray, yellow, orange, or another color, and may be different in color each time the user performs the exercise. The user watches a dot (or other animated object/shape) as it moves around on the scrolling background. The dot appears in different locations on the screen in a different color and/or different size for each appearance, and the user taps the dot wherever it appears. This moving checkerboard is similar to the visual evoked potential image stimulating the striate cortex. The dot that is moving and the user must touch it, which uses the motor cortex. The white checkerboard part changes color to elicit the cone pigment response in the user's retina. This exercise also tests the flow of light from eye to the brain, and whether it is working fast, not so fast, or not at all.

FIG. 3G (Quix 11) and FIG. 3H (Quix 12) are tap the dot (or other animated object/shape) exercises on a set of overlapping shapes, such as rectangles. The rectangles appear as a background on the screen with a colored rectangle in the center and differently colored borders around it. The rectangles/borders change color (e.g., flash from one color to another) and appear to move as the dot also moves around on the rectangles. In FIG. 3G, the rectangles and borders appear in different shades of yellow or blue. This exercise provides color contrast with brighter colors for the yellow cones and blue cones, and retina color cones added with motor cortex of the brain stimulation. This tests the ability to see the gradations of yellow. In FIG. 3H, the rectangles appear in different shades of red and green, which stimulates the red green cones of the retina, along with the motor cortex. This tests the ability to see gradations of red. The user watches a dot (or other animated object/shape) as it moves around on the changing background. The dot appears in different locations on the screen in a different color and/or different size for each appearance, and the user taps the dot wherever it appears.

FIG. 3I (Quix 13) and FIG. 3J (Quix 14) are tap the dot (or other animated object/shape) exercises where the dot appears in different locations on the screen in a different color and/or different size for each appearance. The user's eyes watch the dot as it moves around the screen wherever it appears. The dot is presented on a colorful, busy background of curved or swirled lines. The curved lines are composed of numerous colors (e.g., 2, 3, 4, 5 or more colors, such as blue, green, purple, yellow, pink, or orange), displayed over a solid background (e.g., yellow or blue background). The lines can be different widths, and be irregularly positioned to form a swirled, pattern. Massive color confusion is provided for the 3 cone pigments of the user's retina, and with a dot that is small and hard to find. Retina color cones are added with motor cortex of the brain stimulation. The different colored cones in your retina are competing to see the dots, which are moving in and out of focus. There is also the attentional part of the game--this is stimulating the insula (parietal lobe).

In FIG. 3J, AR is used to view the user's environment in which a grid forms on a surface (e.g., a wall or on a piece of furniture) in the user's own environment. When the user taps the grid, a curved or swirled line pattern with a tap the dot exercise appears replacing the grid on the surface, similar to what is described for FIG. 3I. Again, there is massive color confusion for the 3 cone pigments of the user's retina, and the dot that is small and hard to find with the mobile device camera open for augmented reality. The cerebellum is being stimulated as the user tries to move smartphone around the room to locate the background and the dot. Retina color cones are added with motor cortex of the brain stimulation. The cone pigments are competing with other colors for the user's eyes to find the dot, and there is added texture of the surface inside the user's environment, making the eye and brain work harder. There is also the attentional part of the game—this is stimulating the insula (parietal lobe) (Referance 11).

FIG. 3K (Relax 1) and FIG. 3L (Relax 2) is a set of overlapping shapes, such as rectangles. The rectangles appear as a background on the screen with a colored rectangle in the center and differently colored borders around it. The rectangles/borders change color (e.g., flash from one color to another) and appear to move. In FIG. 3K, the rectangles and borders appear in different shades of yellow, orange, tan, or brown. Yellow is used to convey joy to the user. The changing gradations of color are relaxing to the eye, retina. This exercise senses the ability to see gradations of yellow. The cone cells of the retina are activated. (References 12, 13). In FIG. 3H, the rectangles appear in different shades of green, gray, black, or white. These gray-type tones are used for testing the rod cells in the retina for relative low changes in color intensity, and ability to detect black, white, and gray contrast. These rods are cells in the periphery of the user's retina providing peripheral vision. A different area of the user's retina is being stimulated, the part that is not used often during computer or close work.

FIG. 3M is a scrolling checkerboard (e.g., black) on colored background (e.g., blue) that the user watches. This tests the occipital lobe and the part of the brain next to it, called the striate cortex. It is a reflex and the image goes directly there and forces the brain to be stimulated. It also tests flow of light from the eye to the brain.

FIG. 3N (Relax 4) and FIG. 3O (Relax 5) is an eye relax exercise where the user stares at a black and white or gray pattern that is fuzzy around the border. The stripe pattern changes to various other similar patterns with different sizes or shapes of stripes (e.g., the stripe may change in thickness to thicker or thinner stripes, may change in orientation from between vertical, horizontal or diagonal, and they appear to almost vibrate (vibrating sinusoidal gratings). In terms of the pattern, its movement speed can be varied as well. The directionality of the pattern movement can be varied and not limited to being at 90 degree axis, 180 degree axis, 45 degree axis or 30 degree axis. The brain is stimulated to see the orientation of the stripes and see the edges of the stripes. The occipital lobe is sensitive to line orientation and seeing edges, which is useful for matching fine lines for Vernier Visual acuity (e.g., for micrometer settings, videogaming, or complex robotic surgery). In FIG. 3N, an animated object, such as a dot, moves around the screen over the striped pattern and the user watches the dot move. Gabor patches are used to stimulate the striate cortex and the movement is stimulating the directional cells of the striate cortex. This Gabor patch stimulates the brain and eye perception of directionality. The right front parietal lobe is involved as well. (Referance 14)

In FIG. 3M, the stripes are colored in the center of the pattern (e.g., red colored). Gabor patches, in color, stimulate the striate cortex. The perceived movement is stimulating the directional cells of the striate cortex and frontoparietal lobe. The colors stimulate the retinal cones as well as the brain. (Referance 12). This relaxes the brain after a tough day at the computer.

Visual Pathways and Exercises

The visual pathway extends from the eye, through the brain to end at the lateral geniculate body, and finally at the occipital lobe. However, there are specific targets for the eye exercises:
Retina-Rod function: There are 10 layers of cells, specifically the rod cells to determine contrast sensitivity. To stimulate the rods of the eye, the exercise involves black tones or dark colors versus its closest counterparts. To differentiate the two relative dark colors requires this function of the rods.
Retina-Cone function: Bright colors, red, green, yellow stimulate those pigments in the retina. In addition, small test objects, or using the Amsler Grid (to see the smallest deviations in a straight line) stimulate the cones of the eye.
Fronto parietal lobe: Integrative function, such as the OKN tape (Quix 6, Quix 5), stimulate this portion. This part of the brain contains one of the relay stations of connections of the retina and occipital lobe. In targeting this large part of the brain, this provides an overall “warm up exercise” before targeting specific parts of the brain, e.g., the insula, which is for the attentional aspect of seeing.
Insula: To increase attention span, such as concentration for target practice or microsurgery, the insula can be tested by using confusing colors or objects while the user is asked to find a small target of varying size, shape, speed, or color.
Occipital lobe: This is most often damaged in stroke. To target its brain plasticity, the moving checkerboard or stripes that move in space, or are in different angular positions are effective. This part also tests the cerebellum in discerning spatial movement.
Striate Cortex: This is the site of brain plasticity: This can be tested with slanted horizontal or vertical lines that move. The lines can be varied in terms of size, color and speed of movement. The angular orientation of the lines play a role, whether 90 degrees or 180 degrees, or off-axis. Augmented reality or virtual reality are helpful for this as well. Care should be taken to not trigger nausea and epilepsy in some patients who have problems affecting the occipital lobe and cerebellum.
Cerebellum: This is relevant in rehabilitation for stroke or traumatic brain injury patients where they cannot locate or find a position of an object in space. Augmented Reality helps with this training. Cerebellum is interconnected in exercises involving “touching the dot” or spatial orientation with augmented reality. To test this, the game (Quix 6) can be used.
Motor cortex with visual cortex: Target practice requires this. Adding color confusion to challenge the user creates exercises of increasing difficulty.

The sequence of each of the exercises in a module is relevant since the platform is targeting entire brain and different aspects of the brain as the mobile application continues through a given module. The retina pathways are added into each test as the test progresses. Unlike conventional tests that focus on a particular area of the brain and exercise that area, these exercises follow a particular pathway through different areas of the brain and retina stimulated in a set order for a particular end goal.

### III. Example Visual Stimulation Methods

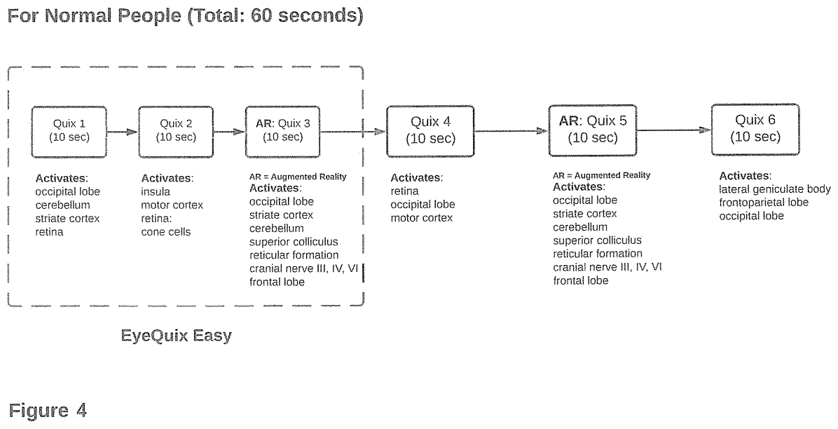

FIG. 4 is a flow chart illustrating a set of exercises of a module and a sequence of those exercises that is specifically designed for “normal” people (e.g., without a particular eye condition), according to some embodiments. FIG. 4 shows that a normal person does 6 exercises (e.g., Quix 1-6) for a time period each (e.g., 10 seconds each for a total time period (e.g., total time for the module of 1 minute of testing). This shows the sequence of exercises and the different parts of the brain or retina that are affected by each exercise. In this example, the user performs the Easy module and performs some additional exercises in a different module.

This sequence is useful for “normal” people because of the particular order of the steps. The user starts in the first exercise (e.g., Quix 1) with the occipital lobe and the cerebellum (e.g., object moving in space), and the retina to see the color of the object. At exercise 2 (e.g., Quix 2), the user uses the insula (for attentional focusing). At exercise 3 (e.g., Quix 3), with augmented reality, the user uses the large part of the brain with the cerebellum playing a large role in user finding the game in space. In the fourth exercise (e.g., Quix 4), the user is back to the retina (smaller part of the visual pathway). In the fifth exercise (e.g., Quix 5), the user is back to the large part of the brain and cerebellum, using the entire visual pathway. Lastly, in the sixth exercise (e.g., Quix 6), the user utilizes reflexes, uncontrolled, and stimulates the part of the brain that is present at the newborn stage of life, primitive vision. This module is organized with a back and forth of total brain and eye, and then is just retina focused, and then finishes with a reflex (e.g., Quix 6).

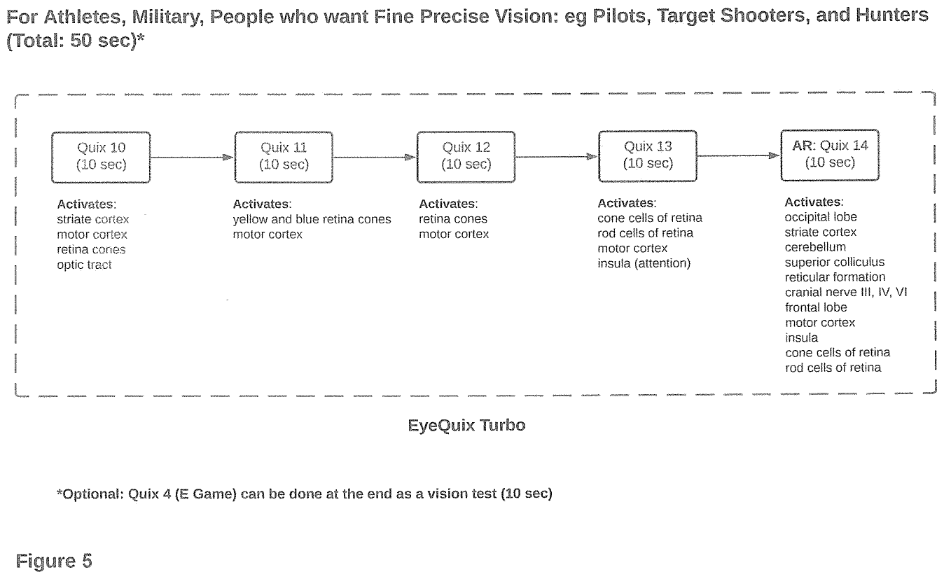

FIG. 5 is a flow chart illustrating a set of exercises of a module and a sequence of those exercises that is specifically designed for precision vision people (e.g., For athletes, military, people who want fine precise vision (e.g., Pilots, Target Shooters, and Hunters), etc., according to some embodiments. If the user wants a harder test or more challenging test (e.g., athletes who want to improve vision, or videogame players who want to see better for a competition), the user can do five exercises (e.g., Quix 10-14) each for a time period (e.g., 10 seconds) for a total time period (e.g., total module testing time of 50 seconds). This shows the sequence of exercises and the different parts of the brain or retina that are affected by each exercise. In this example, the user performs the Turbo module.

The sequence of games begins with a first exercise that is a stimulation to the global brain (warm-up, e.g., Quix 10). And then, proceeds to a second exercise (e.g., Quix 11) involving the yellow cones in the retina (to stimulate the activity that provides the best vision). This strategy is then repeated in the third exercise (e.g., Quix 12), stimulating the red-green cones with a very intense stimulation. In the fourth exercise (e.g., Quix 13), the insula is targeted, which is the portion that mediates attention and ability to focus on a particular task, including providing massive color confusion panels with disappearing dots. The fifth exercise (e.g., Quix 14) ends with the global cerebellum and total brain being targeted by using augmented reality. Another sequence may not be as effective if it does not include an intentionally created a warm-up, a focused stimulation of the part of the eye that gives the best vision (yellow cones), then red and green cones, followed by the color confusion, and ending with a total brain exercise, which altogether work optimally for this particular purpose of providing precision vision exercises.

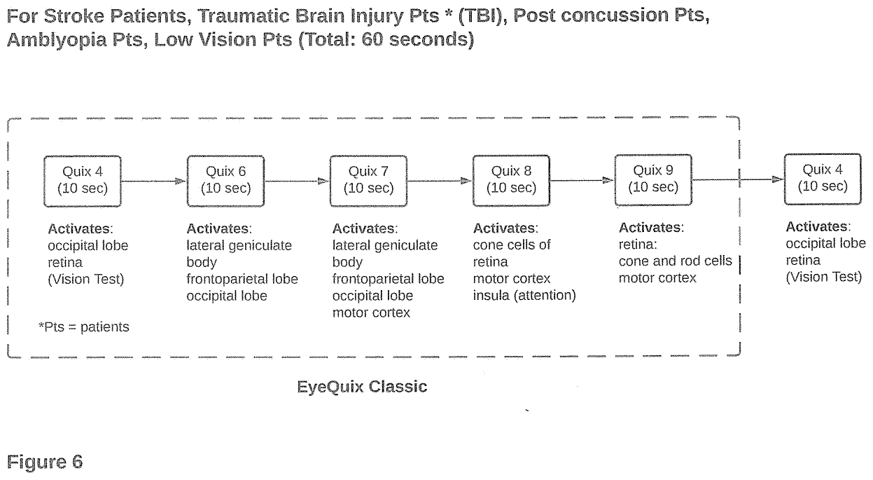

FIG. 6 is a flow chart illustrating a set of exercises of a module and a sequence of those exercises that is specifically designed for people with a vision issue, such as stroke or traumatic brain injury (TBI) patients, post-concussion patients, low vision patients, or amblyopic patients (lazy eye patients), according to some embodiments. The user can do six exercises (e.g., Quix 4-9) each for a time period (e.g., 10 seconds) for a total time period (e.g., total module testing time of 60 seconds). And then the user can repeat test 4 to re-test visual acuity. The test 4 can also be a starting point to initially test visual acuity before doing any additional tests. This shows the sequence of exercises and the different parts of the brain or retina that are affected by each exercise. In this example, the user performs the Classic module.

This sequence is less intense on the eye and retina as the users may have neurological and vision issues. The first exercise, the eyechart provides a good start as a baseline (e.g., Quix 4), reassuring to the patient because the “E” game is familiar. In fact, the “E” chart is familiar internationally in eye clinics. The next exercise (e.g., Quix 6) is the reflex that is tested in newborns, so it provides gentle stimulation that should not be difficult for the user. The next exercise (e.g., Quix 7) targets larger parts of the brain and adds the motor cortex, and the exercise that follows (e.g. Quix 8) adds the attention part of seeing (the insula part of the brain). The module ends with color contrast sensitivity, which targets the retina and the motor cortex (to touch the dot). The sequence is valuable because it does not overstimulate the brain in these patients with known neurological disorders. In amblyopic patients, where there has been a developmental delay in the formation of the visual pathway in one eye versus the normal eye, this sequence targets the need for contrast sensitivity, vision, and attentional looking. If this sequence were performed backward, it may not be as useful because the stroke patient may get easily discouraged and the brain eye pathways have not “warmed up” yet.

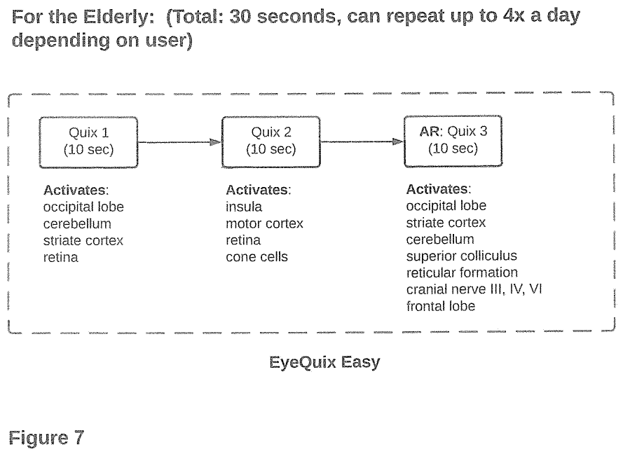

FIG. 7 is a flow chart illustrating a set of exercises of a module and a sequence of those exercises that is specifically designed for elderly people, according to some embodiments. FIG. 6 shows that a normal person does 3 exercises (e.g., Quix 1-3) for a time period each (e.g., 10 seconds each for a total time period (e.g., total time for the module of 30 seconds of testing). These tests can be repeated up to 4 times day. This shows the sequence of exercises and the different parts of the brain or retina that are affected by each exercise. In this example, the user performs the Easy module.

This module allows the use of the total brain in the first exercise (e.g., Quix 1), with the cerebellum being used, and then ramps up to the insula in the second exercise (e.g., Quix 2). In the third exercise (e.g., Quix 3), the total brain eye pathway is being utilized. There are commonly very short for short attention spans with the elderly. If this module were changed, there would be exercises to target total brain and it would be possible to lengthen the time or it would be possible to start with a slower warmup, such as for the stroke patient.

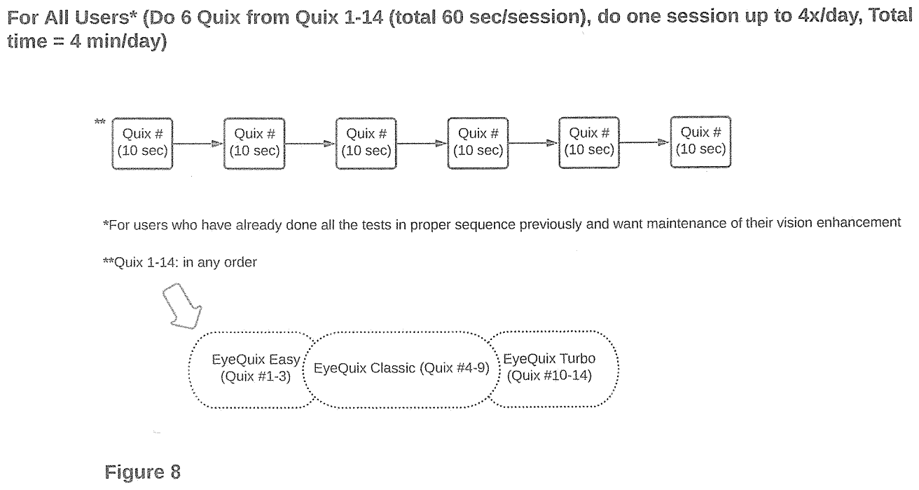

FIG. 8 is a flow chart illustrating a set of exercises of a module and a sequence of those exercises that is specifically designed for all people, according to some embodiments. FIG. 8 shows that for all users who have tried the games in sequence in the previous sessions, the users could use any 6 tests to train vision in any order for one session at e.g., 60 seconds. The user can repeat this 4 times day. The person does 3 exercises (e.g., Quix 1-3) for a time period each (e.g., 10 seconds each for a total time period (e.g., total time for the module of 30 seconds of testing). These tests can be repeated up to 4 times day. This could be some mix of Easy, Classic, and Turbo.

Once the user has tried the preferred module and has improved, the user can choose what the user would like to do. The user can tailor it based on the scores received on the “E” game (e.g., Quix 4) or by how the user performs at the real-world task, e.g., hitting a baseball or target shooting practice. Some of the games, the user may feel vision improvement quickly after the exposure to the game.

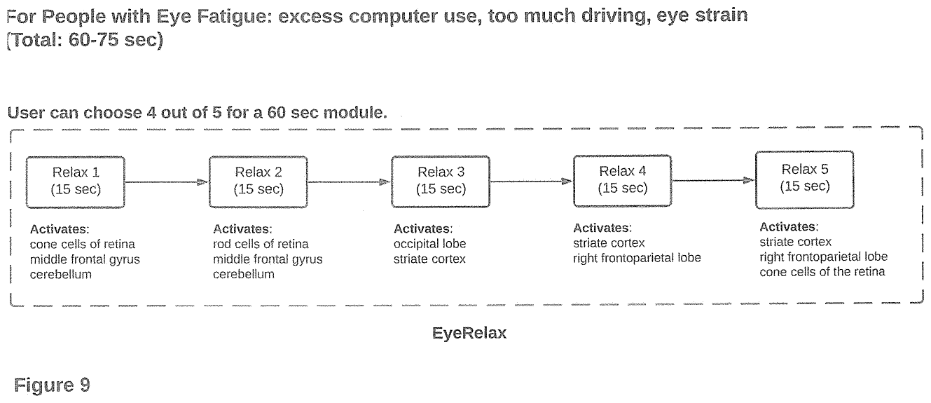

FIG. 9 is a flow chart illustrating a set of exercises of a module and a sequence of those exercises that is specifically designed for people with eye fatigue, according to some embodiments. FIG. 9 shows that for people with eye fatigue, excess computer use, too much driving or eye strain, the users can perform the Relax module, including Relax 1-5. The user can choose 4 out of 5 games for a module of a time period (e.g., 60 second module) or could do all 5 for a longer time period (e.g., 75 seconds). Other variations are also possible.

This sequence for eye fatigue starts with a first exercise (e.g., Relax 1), which is stimulating the cones (pale versions of yellow for mild activation of the yellow cones), and then goes to the second exercise (e.g., Relax 2) that stimulates the rods, (black and white vision and color contrast). The third exercise (e.g., Relax 3) is a reflex and it targets the occipital lobe and striate cortex, so user cannot help but have the brain take over the vision part of seeing. In the fourth exercise (e.g., Relax 4), the striate cortex is stimulated as a reflex. The user cannot help but let the brain take over. Similarly, for the fifth exercise (e.g., Relax 5), there is the added twist of some cone function as the red pigments of the red cones are stimulated. The third, fourth, and fifth exercises are particularly valuable, the second and third serve as warmups. If the user were to not follow the sequence, e.g., to do the fourth and fifth alone, this could cause a headache. Thus, again, the sequence is designed for the particular purpose of addressing eye fatigue, and other sequences may not work as well.

The FIGS. 4-9 show different sequences designed for particular purposes, though these can be revised in certain circumstances to be customized to user or when the user has a slightly different end goal with the exercises.

### IV. Additional Exercise Types

Some exercise types involve a more game-oriented approach where the user interacts with certain animated objects in the environment, where each object is colored, sized, etc. to provide particular stimulation across different visual pathways. In one example, balls are moved around on the screen and the user interacts with the balls. For example, the game can be a sports exercise where the user sees tennis balls exiting a location in the distance and heading toward the user's eyes, possibly bouncing along the way. The user takes an action related to the balls (e.g., ball color selection, ball deflection, ball catching). The user may select balls of a particular color (e.g., red) that are different from the other balls (e.g., yellow), and tap the balls that are the particular color to interact with them (e.g., make them disappear or otherwise modify their movement). The user may tap the screen to deflect all balls regardless of color. The user may move the mobile device to intersect with a ball in order to “catch” each ball as the balls are released toward the user. AR can be used to provide a background of the user's home environment such that the balls appear to come from the user's environment, providing further visual stimulation as the background is moving as the user's mobile device moves, in addition to the bouncing balls. Other types of objects instead of balls can be used, and other types of interactions with the objects can occur. In each case, the exercise is designed to stimulate visual pathways for the user.

### VI. Experimental Data

In an experiment, the Logmar Near Visual Acuity Card (Precision Vision: Woodstock IL) was held at 16 inches to test vision for each eye. The experimenters tested the patient's worse eye according to patient's perception or prior history. A mobile application, according to an embodiment of the visual stimulation platform described herein, was used to perform exercises. A vision test was repeated with the Logmar Near Visual Acuity Card. Inclusion criteria for the experiment included: an ability to use a hand-held smartphone device (e.g., an iPhone 7 or 12 Pro); an age range of 18-90 years old; and a vision range of 20/15 to 20/400. Exclusion criteria for the experiment included: an inability to understand English; an inability to understand instructions; and an inability to use fingers to manipulate the smartphone device or ability to touch the screen. The experiment followed Helsinki Guidelines for ethical research.

The control and patient groups were as follows:

Control Group

| | |
|---|---|
| N= | 38 |
| # of Eyes | 56 |
| Vision Range | 20/15-20/100 |
| Age Range (yrs) | 18-66 |
| Age Average (yrs) | 34.342 |
| Age STD | 16.54 |

Patient Group

| | |
|---|---|
| N= | 38 |
| # of Eyes | 54 |
| Vision Range | 20/20-20/400 |
| Age Range (yrs) | 18-86 |
| Age Average (yrs) | 60.77 |
| Age STD | 15.053 |

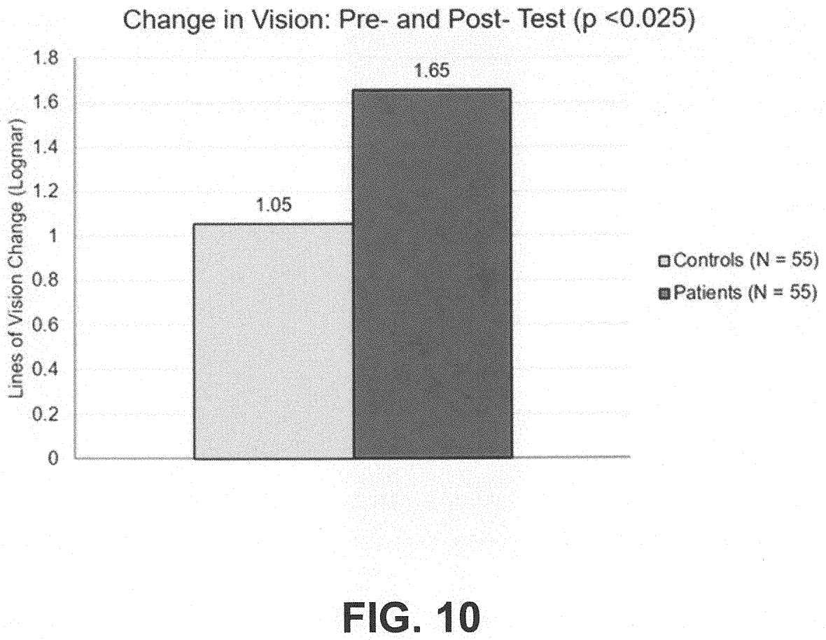

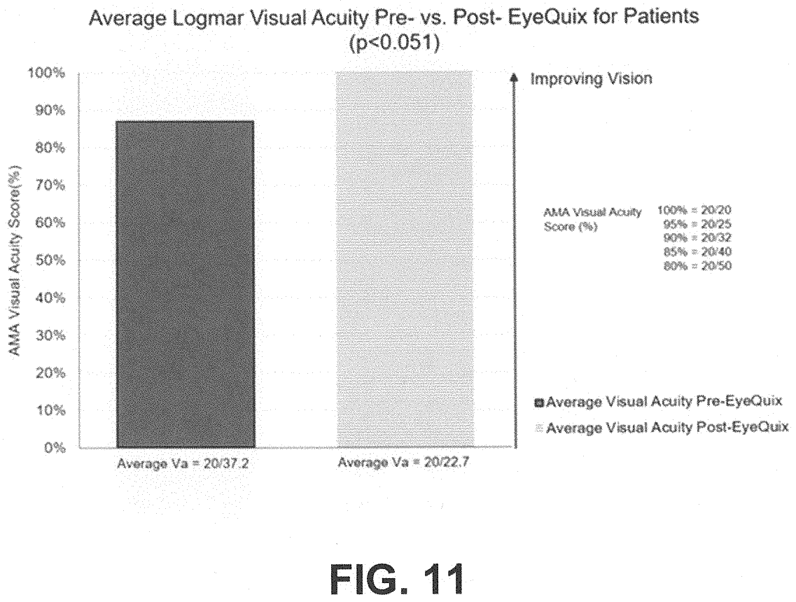

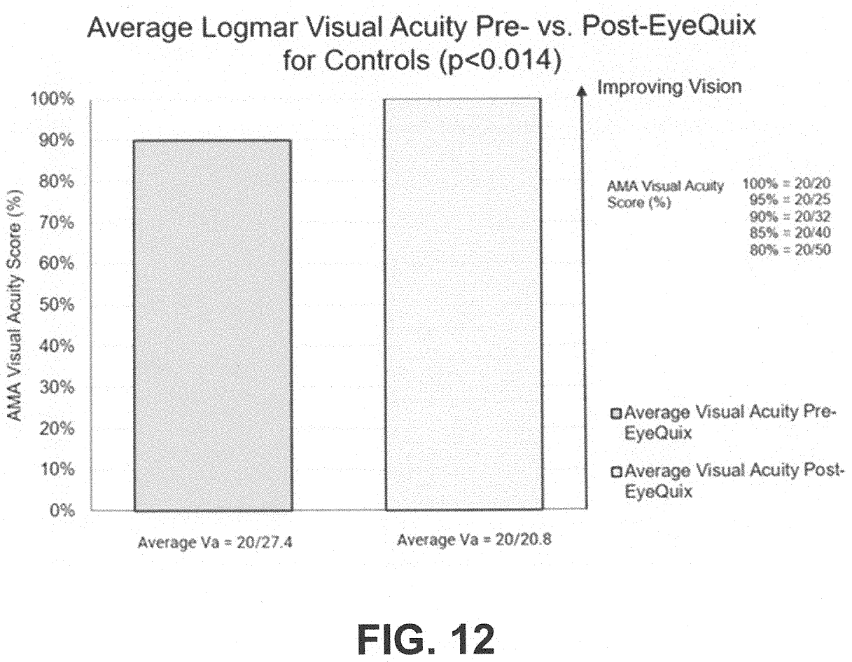

The percentage of eyes in the patient group that had improved visual acuity was 75.45%. FIGS. 10-12 illustrate some additional results of the experiment.

An additional preliminary study was performed, involving 64 eyes, age range 18-83 years. The average age of participants was 44.8 years, the standard deviation of age was 19.69 years, and the median age was 40 years. The average pre-test visual acuity using Logmar vision test (distance) was 45 letters (approximately Snellen visual acuity 20/30). After four exercises were conducted on a visual stimulation system (e.g., exercises 6, 7, 8, and/or 9) , the average post-test visual acuity was 50 letters (approximately Snellen visual acuity 20/25). There was an average gain in 1.98 letters in distance vision (standard deviation was 4.19). This is approximately a 40% gain of vision (5 letters=1 line) . Similarly for near vision, there was a gain of 1.56 letters (standard deviation was 5.98). This is approximately a 30% gain of vision. The Chi-Square test value was: p<0.001. Information about patient group is described below.

Patient Group

| | |
|---|---|
| # of Eyes | 64 |
| Vision Range | 20/20-Hand Motion |
| Age Range (yrs) | 18-83 |
| Age Average (yrs) | 44.8 |
| Age STD (yrs) | 19.69 |

### V. Alternative Embodiments

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable non-transitory medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

The patent or application file contains at least one drawing executed in color. Copies of this patent or patent application publication with color drawing(s) will be provided by the Office upon request and payment of the necessary fee.

VI. References

1. Sincich L C, Park K F, Wohlgemuth M J et al. Bypassing Vl: A direct geniculate input to area MT. Nat Neurosci. 2004; 7:1123-1128.

2. Tamietto M, Pullens p, de Gelder B et al. Subcortical connections to human amygdala and changes following destruction of the visual cortex. Curr Biol. 2012; 22:1449-1455.

3. Celine PErez, Sylvie Chokron. Rehabilitation of homonymous hemianopia: insight into blindsight. Front Integr Neurosci. 2014: https://doi.org/10.3389/fnint.2014.00082. Accessed 24 Apr. 2021.

4. Bridge H, Leopold D A, Bourne J A. Adaptive pulvinar circuitry supports visual cognition. Trends Cogn Sci 2015. https://doi.org/10.1016/j.tics.2015.10.003, Accessed 24 Apr. 2021

5. Ajina S, Pestilli F, Rokan A et al. Human Blindsight is mediated by an intact geniculo-extrastriate pathway. Elife. 2015;A https://doi.org/10.7554/eLife.08935.001, accessed 24 Apr. 2021.

6. Leh S E, Johansen-Berg, H, Ptito A. Unconscious vision: new insights into the neuronal correlates of blindsight using diffusion tractography. Brain 2006; 129:1822-1832.

7. Tamietto M, Morrone M C. Visual Plasticity: Blindsight bridges anatomy and function in the visual system. Curr Biol 2016;26(2): 70-73.

8. Sabel B A, Henrich-Noack P, Fedorow A et al. Vision restoration after brain and retina damage: The residual vision activation theory. Prog Brain Res. 2011;192:199-262.

9. Furman J M. Optokinetic Nystagmus. In Encyclopedia of the Neurological Sciences (eds Aminoff M J, Daroff R B, Academic Press. https://doi.org./10.016/B978-0-12-385157-4.00150-0. Accessed 24 Apr. 2021.

10. Dieterich M, Bucher S F, Seelos K C et al. Horizontal vertical optokinetic stimulation activates visual motion-sensitive, ocular motor and vestibular cortex areas with Right hemispheric dominance. Brain 1998; 121(8): 1479-1495.

11. Gang D, He H, Liu D, et al. Enhanced functional connectivity and increased gray matter volume of insula related to action video game playing. Scientific Reports. 2015; 5: 1-7.

12. Bird C, Berens S, Homer A et al. Categorical encoding of color in the brain. PNAS. 2014; 111:4590-4595.

13. Clifford A, Franklin A, Holmes A et al. Neural correlates of acquired color category effects. Brain and Cognition. 2012;80:126-143.

14. Fiori F, Candidi M, Acciarino A et al. The right temporoparietal function plays a causal role in th maintaining the internal representation of verticality. J Neurophysiology 2015.

15. Yang X, Lu L, Li Q et al. Altered spontaneous brain activity in patients with strabismic amblyopia: a resting state fMRI study using regional homogeneity analysis. Exp Therapeutic Med. 2019. https://doi.org/10;3892/etm.2019.8038. Accessed 26 Apr. 2021.

16. Dai P, Zhang J, Wu T et al. Altered spontaneous brain activity of children with unilateral amblyopia: a resting state fMRI study. Neural Plasticity. 2019. https://doi.org.10.1155/2019/3681430. Accessed 26 Apr. 2021.

17. Basgoze Z, Mackey A P, Cooper E A. Plasticity and Adaptation in Adult Binocular Vision. Curr Biol 2018; 28:1406-1413. https://doi.org810.1152/in.00289. Accessed. 24 Apr. 2021.

What is claimed is:

1. A method comprising:
receiving, in a mobile application on a client device, a selection from a user of a module of eye exercises to stimulate a visual pathway of the user;
providing, for display to the user on the mobile application, a first eye exercise in an ordered sequence of exercises, the first eye exercise comprising one or more screens showing an animated display for the user to view for a time period less than a threshold time, the animated display having one or more of a color, movement, or pattern that is designed to stimulate a first visual pathway of the brain or the retina of the user;
receiving an indication that the user has completed the first eye exercise;
following receipt of the indication of completion of the first eye exercise, providing for display to the user on the mobile application, a second eye exercise in an ordered sequence of exercises, the second eye exercise comprising one or more screens showing an animated display for the user to view for a time period less than a threshold time, the animated display having one or more of a color, movement, or pattern that is designed to stimulate a second visual pathway of the brain or the retina of the user;
receiving an indication that the user has completed the second eye exercise;
upon the user completing the module of eye exercises, confirming to the user the completion of the module;
wherein at least one of the first and second eye exercises comprises an interactive portion for the user to interact with one or more items on the screen to test the motor cortex of the user, and
wherein the ordered sequence of exercises is ordered specific to a particular end goal of the user in conducting the eye exercises.

2. The method of claim 1, further comprising:
providing for display to the user on the mobile application a third eye exercise in an ordered sequence of exercises, the third eye exercise comprising one or more screens showing an animated display for the user to view for a time period less than a threshold time, the animated display having one or more of a color, movement, or pattern that is designed to stimulated a third visual pathway of the brain or the retina of the user.

3. The method of claim 1, further comprising:
providing for display to the user on the mobile application a plurality of additional eye exercises in an ordered sequence of exercises, the additional eye exercises each comprising one or more screens showing an animated display for the user to view for a time period less than a threshold time, each of the animated displays having one or more of a color, movement, or pattern that is designed to stimulated a particular visual pathway of the brain or the retina of the user.

4. The method of claim 1, wherein at least one of the first and second exercises comprises an augmented reality exercise in which the client device of the user displays a pattern overlaid on an augmented reality view of an environment of the user.

5. The method of claim 4, wherein an animated eye chart is displayed over the augmented reality view of the environment of the user.

6. The method of claim 4, wherein a scrolling checkerboard pattern is displayed over the augmented reality view of the environment of the user.

7. The method of claim 6, wherein an animated object moves to different locations on the pattern each time the user taps on the animated object.

8. The method of claim 5, wherein the pattern comprises a plurality of curved lines of different colors and widths, and wherein an animated object moves to different locations on the pattern each time the user taps on the animated object.

9. The method of claim 1, wherein at least one of the first and second exercises comprises a pattern composed of three or more different colors on the display.

10. The method of claim 9, wherein an animated object moves to different locations on the pattern each time the user taps on the animated object.

11. The method of claim 10, wherein the pattern comprises a plurality of curved lines of different colors and widths.

12. The method of claim 1, wherein the at least one of the first and second exercises comprises a checkerboard pattern composed of at least two different colors.

13. The method of claim 12, wherein an animated object moves to different locations on the pattern each time the user taps on the animated object.

14. The method of claim 1, wherein at least one of the first and second exercises comprises a box surrounded by a plurality of different colored borders provided on the display, the box and the borders changing color as the user views the display.

15. The method of claim 14, wherein an animated object moves to different locations on the display each time the user taps on the animated object.

16. The method of claim 1, wherein at least one of the first and second exercises comprises a pattern of lines on the display, where border around the lines is blurred.

17. The method of claim 16, wherein an animated object moves to different locations on the display while the user views the lines on the display.

18. The method of claim 16, wherein the center of the display comprises a different color than the lines on the display.

19. The method of claim 16, wherein the pattern of lines on the display change at intervals, including changing to lines of a larger or smaller width, or lines presented in a different orientation than previously displayed.

20. A computer program product comprising a non-transitory computer readable storage medium having instructions encoded thereon that, when executed by one or more processors, cause the one or more processors to perform steps comprising:
receiving, in a mobile application on a client device, a selection from a user of module of eye exercises to stimulate a visual pathway of the user;
providing for display to the user on the mobile application, a first eye exercise in an ordered sequence of exercises, the first eye exercise comprising one or more screens showing an animated display for the user to view for a time period less than a threshold time, the animated display having one or more of a color, movement, or pattern that is designed to stimulate a first visual pathway of the brain or the retina of the user;
receiving an indication that the user has completed the first eye exercise;
following receipt of the indication of completion of the first eye exercise, providing for display to the user on the mobile application, a second eye exercise in an ordered sequence of exercises, the second eye exercise comprising one or more screens showing an animated display for the user to view for a time period less than a threshold time, the animated display having one or more of a color, movement, or pattern that is designed to stimulate a second visual pathway of the brain or the retina of the user;
receiving an indication that the user has completed the second eye exercise;
upon the user completing the module of eye exercises, confirming to the user the completion of the module;
wherein at least one of the first and second eye exercises comprises an interactive portion for the user to interact with one or more items on the screen to test the motor cortex of the user, and
wherein the ordered sequence of exercises is ordered specific to a particular end goal of the user in conducting the eye exercises.

21. A computer-implemented system comprising:
a processor;
a computer readable medium storing code that when executed by the processor causes the processor to perform steps comprising:
receiving, in a mobile application on a client device, a selection from a user of module of eye exercises to stimulate a visual pathway of the user;
providing for display to the user on the mobile application, a first eye exercise in an ordered sequence of exercises, the first eye exercise comprising one or more screens showing an animated display for the user to view for a time period less than a threshold time, the animated display having one or more of a color, movement, or pattern that is designed to stimulate a first visual pathway of the brain or the retina of the user;
receiving an indication that the user has completed the first eye exercise;
following receipt of the indication of completion of the first eye exercise, providing for display to the user on the mobile application, a second eye exercise in an ordered sequence of exercises, the second eye exercise comprising one or more screens showing an animated display for the user to view for a time period less than a threshold time, the animated display having one or more of a color, movement, or pattern that is designed to stimulate a second visual pathway of the brain or the retina of the user;
receiving an indication that the user has completed the second eye exercise;
upon the user completing the module of eye exercises, confirming to the user the completion of the module;
wherein at least one of the first and second eye exercises comprises an interactive portion for the user to interact with one or more items on the screen to test the motor cortex of the user, and
wherein the ordered sequence of exercises is ordered specific to a particular end goal of the user in conducting the eye exercises.

22. A computer-implemented system comprising:
a processor;
a computer readable medium storing code that when executed by the processor causes the processor to perform steps comprising:
in response to a selection from a user of a module of eye exercises to stimulate a visual pathway of the user, receiving, from a server at a mobile application on a client device, a first eye exercise in an ordered sequence of exercises, the first eye exercise comprising one or more screens showing an animated display for the user to view for a time period less than a threshold time, the animated display having one or more of a color, movement, or pattern that is designed to stimulate a first visual pathway of the brain or the retina of the user;
receiving an indication that the user has completed the first eye exercise;
following receipt of the indication of completion of the first eye exercise, receiving, from a server at a mobile application on a client device, a second eye exercise in an ordered sequence of exercises, the second eye exercise comprising one or more screens showing an animated display for the user to view for a time period less than a threshold time, the animated display having one or more of a color, movement, or pattern that is designed to stimulated a second visual pathway of the brain or the retina of the user;
receiving an indication that the user has completed the second eye exercise;
upon the user completing the module of eye exercises, confirming to the user the completion of the module;
wherein at least one of the first and second eye exercises comprises an interactive portion for the user to interact with one or more items on the screen to test the motor cortex of the user, and
wherein the ordered sequence of exercises is ordered specific to a particular end goal of the user in conducting the eye exercises.
